(19) United States
(12) Patent Application Publication
(10) Pub. No.: US 2024/0269097 A1
(43) Pub. Date: Aug. 15, 2024

(54) Rapid Onset Ketosis Enabled by Bioidentical D-BetaHydroxyButyric Acid Systems, Compounds and Methods for Hunger Reduction and Enhanced Fasting

(71) Applicant: American Ketone LLC, Sheridan, WY (US)

(72) Inventor: Heidi H Hynes, Seattle, WA (US)

(21) Appl. No.: 18/419,229

(22) Filed: Jan. 22, 2024

Related U.S. Application Data
(63) Continuation-in-part of application No. 17/002,691, filed on Aug. 25, 2020.
(60) Provisional application No. 63/481,172, filed on Jan. 23, 2023.

(51) Int. Cl. A61K 31/19; A61K 31/13; A61K 31/198; A61K 31/51; A61K 35/74; A61K 38/39; A61P 3/00

(52) U.S. Cl. CPC A61K 31/19; A61K 31/13; A61K 31/198; A61K 31/51; A61K 35/74; A61K 38/39; A61P 3/00

(57) ABSTRACT

The claimed uses of bioidentical free D-BetaHydroxyButyric Acid as a method (1303), system (1411) and compound for hunger reduction are novel, safe and not anticipated by the body of research spanning well over 75 years. Overall, no adverse findings have been reported within cell, animal and human safety studies at high dose levels with D-BetaHydroxyButyric acid which supports the safety and efficacy of the claimed uses at the anticipated usage levels for the disclosed applications.

### CROSS-REFERENCE TO RELATED APPLICATIONS

This patent application is related to and claims priority from previously filed provisional application Ser. No. 63/481,172 filed on Jan. 23, 2023. This application additionally is a continuation in part of Ser. No. 17/002,691 filed Aug. 25, 2020.

### TECHNICAL FIELD

The claimed invention has applicability in consumer dieting. With greater particularity, the claimed invention is relevant to diet and hunger reduction. With still greater particularity, the claimed invention is directed at novel uses of the bioidentical form of the naturally occurring ketone body D-BetaHydroxyButyric Acid for hunger reduction while dieting and fasting with related systems and methods.

### BACKGROUND OF THE INVENTION

While health benefits from fasting are known, unaided fasting or ‘water fasts’ are simply extremely difficult to engage in without physiological aid. From a pharmacological perspective, GLP 1 agonists (glucagon like peptide 1) have gained popularity to reduce hunger for weight loss. GLP1, the target of GLP1 agonists, was discovered through bariatric surgery. In addition, it was noteworthy that many bariatric surgery patients are cured of Type 2 diabetes. GLP1 drugs act like a hormone which is naturally released by the incretin system in the intestines in response to food. They provide a strong signal in the brain for brown fat tissue activation and to the brain to enhance satiety and reduce hunger. GLP1 is an amino acid that researchers tracked for its increase in the intestines due to reduced food intake and periods of fasting. As the stomach is much smaller after surgery, the gastric emptying time is slower so the period of time between food intake is extended.

Semaglutide is the first GLP1 hunger reduction drug to come on the market. Semaglutide improves the efficiency of incretin function by activating GLP-1 receptors. It acts by numerous mechanisms like augmented insulin secretion (glucose-dependent), inhibition of glucagon release and suppressed hepatic gluconeogenesis; thereby reducing both fasting glucose as well as postprandial glucose. While the side effects are known for Type 2 diabetes, this product is now being marketed for obesity at much higher doses. As the safety of prolonged use at much higher doses remains unclear, expanding the use of this category of drugs to more than 70% of the population that is overweight and obese along with children may have serious long term health consequences.

In addition to pharmacological solutions to reduce hunger, known ketogenic supplements have been tried to reduce hunger with limited results. Of the two previously known ketogenic supplements used to reduce hunger, using the chemically synthetic ‘ketone ester’ to reduce hunger is accompanied by substantial drawbacks including extremely challenging (or outright horrible) taste profile with unacceptable levels of vomiting, high cost and unknown liver metabolism impact. In addition, the commercially known ketone salts do not reduce hunger in a meaningful or significant way. Consequently, better ways are needed to utilize exogenous ketones to reduce hunger during fasting and hunger reduction diet scenarios.

### BRIEF SUMMARY OF THE INVENTION

Overview of Disclosure: the claimed invention utilizes bioidentical D-BetaHydroxyButyric Acid (bioidentical D-BHB) to reduce hunger in lieu of the new and popular GLP1 drugs that have entered the market. It is an objective of the claimed invention to reduce hunger without side effects or long term health consequences. As the dosage of GLP1 drugs such as Semaglutide when sold for weight loss is significantly higher than the dose sold for type 2 diabetes, long term health consequences for GLP1 drugs when used for hunger reduction may be substantial. Moreover, it is believed that continual administration over a lifetime is required otherwise reversion to an obese state with increased weight gain. Finally, muscle wasting and possible bone density loss may occur in conjunction in weight loss. Consequently, there is a clear and compelling need for hunger solutions without long term pharmacological risks or challenges.

Natural free D BHB is the same molecule made endogenously by your body during fasting, it is an efficient fuel source and a power signaling molecule, on average our 10 g of free DBHB raises ketones by 1.5 mmolL such as one could expect after 2 days of fasting providing similar hunger reduction. For those raised on a diet in excess of 100 gram carbohydrate per day who have never experienced ketosis (Cahill 1967), our product can be a useful tool to introduce a lifestyle defined by better metabolic wellness.

The claimed system, method and compounds utilize a novel form of exogenous ketone D-BetaHydroxyButyric Acid which is administered in ‘free acid form’ which is ‘D only’ and in the complete absence of a salt or ester chemical component. The claimed bioidentical free D-BHB administered exogenously for hunger reduction mimics the effects of fasting and reduces hunger. Bioidentical D-BHB reduces hunger and caloric intake and eliminates the desire for snacking throughout the day. Study participants have found that they have reduced hunger in the presence of food cues, social cues and under times of stress along with an increased focus and energy throughout daily activities.

The novel benefits of D-Beta HydroxyButyric Acid arise from exogenous administration in diluted drink or bolus format to reduce hunger. The first section of the disclosure addresses the enabling benefits of exogenous D-Beta HydroxyButyric Acid administered in the absence of a chemical salt or ester configuration, followed by an identity section further detailing chemical identity of D-BHB followed by blood level ketosis data according to the claimed invention. Ketosis may be naturally induced through strict diet control or fasting, induction of rapid ketosis utilizing the bioidentical form of the ketone body D-BetaHydroxyButyric Acid optimizes ketone levels while greatly reducing hunger. While a variety of artificial exogenous ketone supplementation has been known and tried in a variety of forms over the past fifty years, both the chemically created ‘ketone salt’ and synthetic ‘ketone ester’ are non-natural solutions with direct limitations and sub-optimal consequences when used for hunger reduction. Ketone chirality matters, and the often “D+L” ketone salts exhibit high salt load coupled with impaired ketosis levels. Moreover, the synthetic ‘ketone ester’ is an alcohol, an artificial, never before seen in man engineered construction of organic chemistry which requires subsequent stomach and liver processing before becoming bioavailable. Consequently, there has been a long felt need and compelling reason to utilize bioidentical D-BetaHydroxyButyric Acid for hunger reduction. Applicant's exogenous D-Beta HydroxyButyric Acid administered in the absence of a chemical salt or ester and the subject of ClinicalTrials.gov identifier NCT05584371. This is the first time the ‘bioidentical’ form of D-Beta HydroxyButyric Acid in the absence of a ‘ketone salt’ or ‘ketone ester’ has been tested in a clinical setting with positive results.

The bioidentical form of D-BetaHydroxyButyric Acid is rapidly absorbed and immediately bioavailable which is in direct contrast to the moderately ketogenic ketone salts and ketone ester compounds which require stomach disassociation followed by liver processing. The greatly improved bioavailability of free D-BetaHydroxyButyric Acid improves reduced hunger outcomes. In addition to known glucose reduction attributes, the novel and hereby disclosed prophylactic glucose spike protective effects have substantial protective effects for optimizing metabolic interventions for hunger reduction.

TABLE 1
Free D-β-HydroxyButyric Acid (D-BHB)
Free D-BHB is both mitochondrial energy source and regulatory
compound with the fastest bioavailability and efficacy vs synthetic ester
and chemical salt.
Unexpected cognitive bioenergenics and neuroprotection benefits.
Free D-BHB is a rapid release format.
Free D-BHB is safe and non-toxic.
Effects: Regulatory, Direct Mitochondrial Energy
Metabolism: Rapid, minutes
Use in Humans: Ancient
Induces Ketoacidosis: No
Chemical Structure:

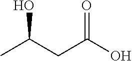

The rationale for a bioidentical D-BHB health regime is primarily supported by the hunger reduction properties of bioidentical D-BHB, high ketone levels generated by the same coupled with high bioavailability without salt load or liver metabolism requirements. As the bioidentical ‘free’ form. The bioidentical ‘free’ D-BHB form is immediately bioavailable for rapid metabolism which is materially distinct from the ‘ketone salt’ compounds (often D+L racemic) chemically joined to a salt or the synthetic ‘ketone ester’ compounds which are alcohol based and must be processed in the stomach and liver before releasing the ketone contained therein.

### BRIEF DESCRIPTION OF THE SEVERAL VIEWS OF THE DRAWING(S)

The accompanying drawings are included to better illustrate exemplary embodiments of the claimed invention.

FIG. 1 is a structural illustration of endogenous ketones.

FIG. 2 is a structural illustration of chemical structures of bioavailable free D-BetaHydroxyButyric Acid, ketone ester and ketone salt.

FIG. 3 is a graphical illustration of endogenous ketone levels by age.

FIG. 4 is a graphical illustration of endogenous ketone brain metabolism.

FIG. 5 is a graphical illustration of comparative exogenous ketone blood levels.

FIG. 6 is a graphical illustration of anti-inflammatory metabolism of ketones.

FIG. 7 is a graphical illustration of free D-BetaHydroxyButyric Acid preparation for hunger.

FIG. 8 is a graphical illustration of big dipper vs little dipper theory of glucose regulation and hunger.

FIG. 9 is a graphical illustration of D-BHB exogenous ketone blood levels.

FIG. 10 is a graphical illustration of D-BHB exogenous ketone blood levels.

FIG. 11 is a graphical illustration of continuous monitoring of body glucose levels.

FIG. 12 is a graphical illustration of continuous monitoring of body ketone levels resulting in improved hunger reduction due to enhanced D-BHB levels according to the claimed invention.

FIG. 13 is a schematic illustration of a preferred D-BHB hunger management method.

FIG. 14 is a schematic illustration of a preferred D-BHB hunger management system.

### DETAILED DESCRIPTION OF THE INVENTION

Introduction: The claimed invention protects against hunger and enhances diet outcomes by exogenously administering bioidentical D-BetaHydroxyButyric Acid.

### I. D-Beta HydroxyButyric Acid Compound Disclosure and Anti-Hunger Applications.

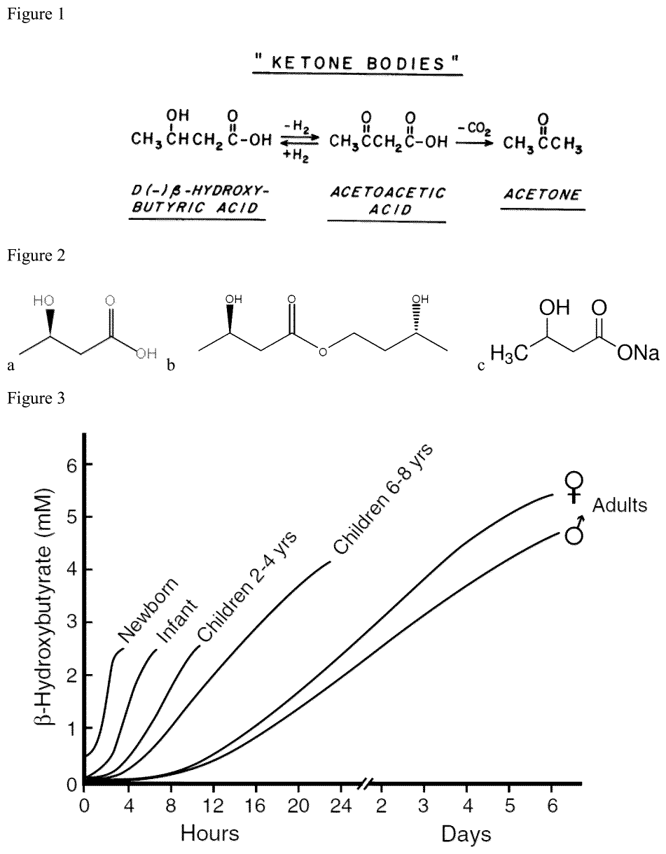

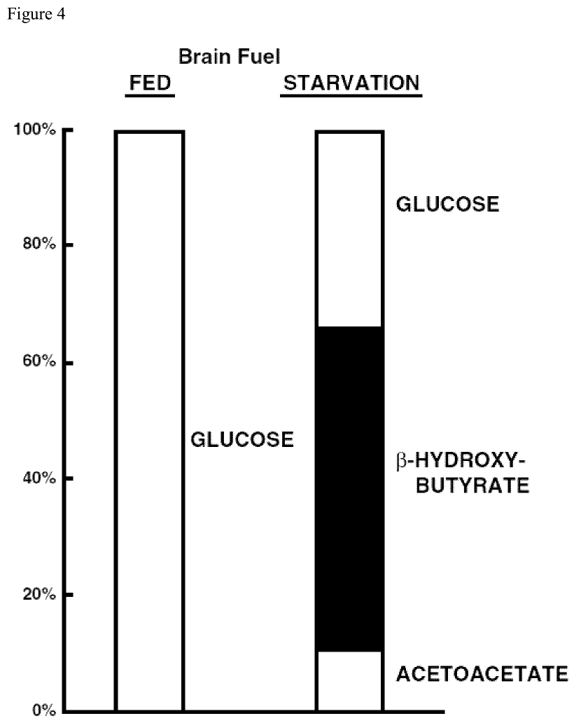

FIG. 1 is a structural illustration of endogenous ketones. The illustration of the endogenously produced ketone bodies is from FIG. 4 of the Harvard Medical School Professor/Dr. George F. Cahill, Jr.'s lecture entitled “Ketosis” given over fifty years ago. Dr. Cahill's foundational work provides clear and concise understanding of ketosis to aid in the understanding of the claimed invention. The FIG. 2 (a) chemical formula identifies the claimed bioidentical ‘free’ form of the exogenous D-Beta HydroxyButyric Acid administered in the absence of a chemical salt or ester and is the subject of ClinicalTrials.gov identifier NCT05584371. The claimed exogenously administered bioidentical free D-BetaHydroxyButyric Acid is chemically identical to the D-BetaHydroxyButyric Acid identified in FIG. 1. Greater detail is hereby provided in the submitted BMJ Nutrition, Prevention & Health journal article. To be clear, this publication is the first reported clinical trial of bioidentical free D-BetaHydroxyButyric acid administered exogenously. The intervening fifty years between the Jeremiah Metzger Lecture given in Boston in 1972 and the present clinical trial and disclosure do not detail bioidentical free D-BetaHydroxyButyric Acid but instead disclose a wide variety of precursors, chemical combinations and not naturally occurring synthetic products which are not bioidentical in their administered form.

FIG. 2 is a structural illustration of bioidentical ketone and chemically synthetic chemical ketone structures. FIG. 2(a) depicts bioidentical free BetaHydroxyButyric Acid. FIG. 2(b) depicts the synthetic ketone ester and FIG. 2 (c) shows the chemical ketone salt. Put simply, bioidentical free D-Beta HydroxyButyric Acid offers direct mitochondrial energy resulting in optimized wellness outcomes when administered in the absence of a chemical salt or ester configuration. To date, exogenous ketone supplementation has been attempted as an aide in following the ketogenic diet. Unfortunately, ketone salts result in an abnormally high salt intake rate and the newly created (R)-3-hydroxybutyl (R)-3-hydroxybutyrate (D-β-hydroxybutyrate ester) or ‘ketone ester’ is a never before seen in man synthetic creation with bioavailability and other long-term concerns. FIG. 2(a) illustration of the chemical form of bioidentical free D-BetaHydroxyButyric Acid is also distinguishable from ketone salts and ketone esters owing to its immediate bioavailability and absence of chemically joined salt or ester bond.

FIG. 3 is a graphical illustration of endogenous ketone levels by age and FIG. 4 is a graphical illustration of endogenous ketone brain metabolism from Dr./Prof Cahill's journal article “Fuel Metabolism in Starvation” provided by Information Disclosure Statement. FIG. 3 details the time to ketosis in healthy adults vs children measured in hours or days. D-BetaHydroxyButyric Acid is a naturally occurring ketone which is produced by the human body. The ketone plays a crucial role during development and throughout the healthy human lifespan. It is produced naturally during fasting (detailed by Cahill's graph above) as well as through the use of ketogenic diets. As Cahill observed, “Due to its use by brain, D-β-HydroxyButyric acid not only has permitted man to survive prolonged starvation, but also may have therapeutic potential owing to its greater efficiency in providing cellular energy in ischemic states such as stroke, myocardial insufficiency, neonatal stress, genetic mitochondrial problems, and physical fatigue.” Cahill's early insights on D-β-HydroxyButyric acid are now through Applicant's clinical trial efforts becoming proven in reality. Applicant's clinical trials including ClinicalTrials.gov identifier NCT05584371 are the first documented clinical applications of free D-BetaHydroxyButyric Acid as further detailed in the provided Dr.Prof. Soto-Mota IDS journal article. FIG. 4 details the fact that ketones in general, and D-BetaHydroxyButyric Acid in particular is the preferred fuel in the human brain. The time to ketosis levels are relevant in the context of the instant application in that creating a rapid 1.5-2.5 mmol/L increase in ketone levels is simply not practical through fasting or diet when rapidly needed in a rapid hunger protection context.

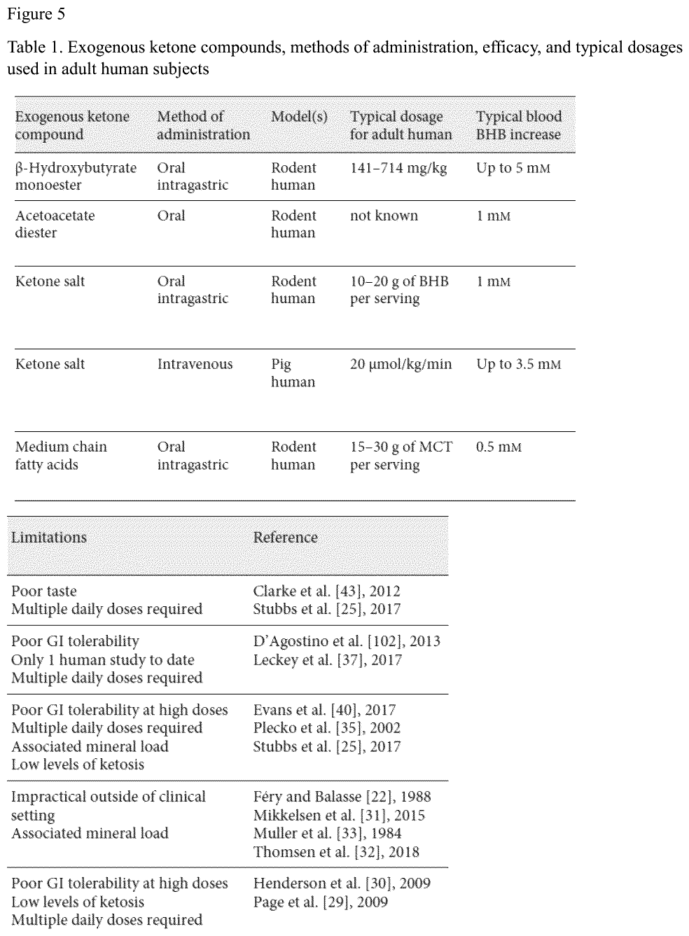

FIG. 5 is a graphical illustration of comparative exogenous ketone blood levels. FIG. 5 depicts Table 1 from “Exogenous Ketone Bodies as Promising Neuroprotective Agents for Developmental Brain Injury” and details the significant limitations among the variety of previously known exogenous salt and ester ketone compounds. Ketone salts are often racemic D+L mixtures which rarely provide greater than 1 mmol/L ketone increase along with substantial salt load rendering it unsuitable for long term daily administration. Similarly, while the synthetic ‘ketone ester’ does provide a higher ketone level response, its highly unpalatable taste renders it highly unsuitable for use in consumer hunger reduction applications. FIG. 5 does not include free D-BetaHydroxyButyric Acid due to the fact that ClinicalTrials.gov identifier NCT05584371 and the provided BMJ Nutrition, Prevention & Health journal article is the first instance of exogenous free D-BetaHydroxyButyric Acid journal publication. Put simply, there has been a longstanding need for a salt free, bioidentical solution to rapid ketosis without alcohol or need for further liver metabolism to rapidly increase ketones to a level relevant for hunger protection.

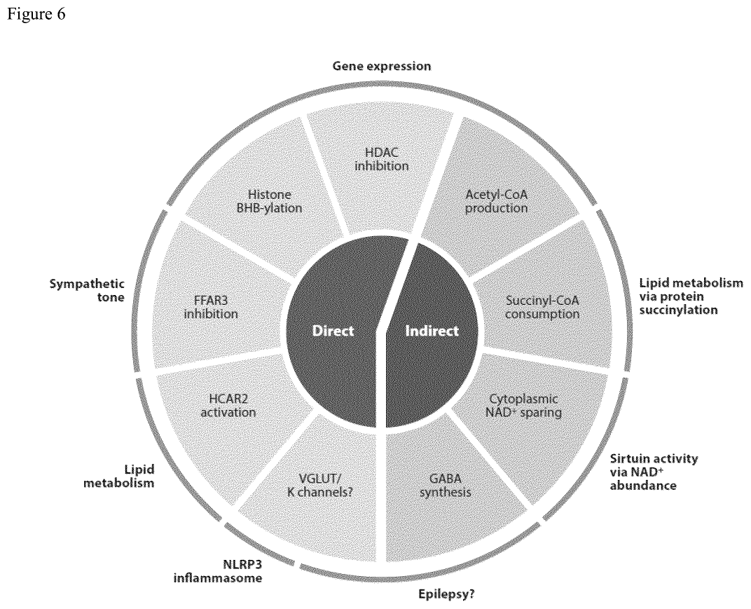

FIG. 6 is a graphical illustration of brain metabolism of ketones from Prof Newman and Prof Verdin's journal article “β-Hydroxybutyrate:A Signaling Metabolite” figure number 2. This figure details “Schematic of direct and indirect signaling functions of the ketone body BHB. Indirect signaling functions require catabolism to other molecules, whereas direct signaling functions are actions of BHB itself.” Applicants respectfully submits that Professors Newman and Verdin detail the starting point of the wide variety of benefits of BetaHydroxyButyric Acid extending to hunger reduction.

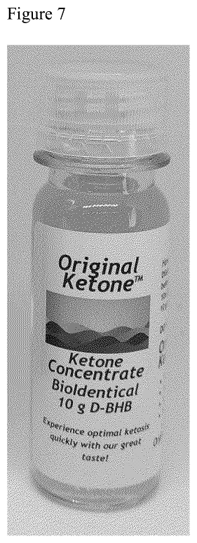

FIG. 7 is a graphical depiction of an illustrative example of free D-BetaHydroxyButyric Acid preparation. In terms of hunger reduction compounds incorporating free D-BetaHydroxyButyric Acid, while preferred embodiments are provided as 5 g-30 g format, larger and smaller amounts may be provided while maintaining the spirit and scope of the invention. In a preferred embodiment, free D-BetaHydroxyButyric Acid is combined and provided in conjunction with a ketogenic complementary nutritional supplement. In an illustrative embodiment, the ketogenic complementary nutritional supplement is selected from akkermansia, taurine, creatine and thiamine. The primary component to be avoided is any kind of sugar, whether in the form of sucrose, glucose, fructose or the like. Additionally, an alternate preferred embodiment includes a palatability agent incorporating sugar free sweetener such as stevia or allulose as well as natural fruit essence distillate as a palatability enhancer.

The depicted preparation of D-BHB free acid is designed for rapid and easy administration for hunger reduction. While 10 g D-BHB free acid is depicted a wide range of D-BHB administration is anticipated according to the claimed invention. Illustrative embodiments utilizing bioidentical free D-BHB in anticipation of food cues, social cues and stress along with anticipation of hunger an effective shield against hunger for 4-6 hours are detailed as follows. Obese BMI individuals may dilute 15-16 grams of natural free dBHB in 700 ml of water and enjoy 30 minutes prior to food, social or stress cues or in anticipation of hunger. Overweight BMI individuals can dilute 8-10 grams of natural free dBHB in 700 ml of water and enjoy 30 minutes prior to food, social or stress cues or in anticipation of hunger. Normal weight individuals may dilute 4-5 grams of natural free dBHB in 500 ml of water and enjoy prior to food, social or stress cues or in preparation for a fast. In additional embodiments, blood or interstitial fluid glucose and ketone levels may be tested to monitor glucose ketone index for iterative improvement of lifestyle habits extending and enhancing metabolic wellness. In terms of absorption, while peak plasma levels are reached at 30-45 minutes post drink, our product is immediately available upon drinking as unlike monoesters, diesters and salts it does not require the liver to process. The claimed compound is not bound by esters or salt and is immediately absorbed in the upper intestines with continued absorption as it is further digested. Bioidentical free D-BHB bioavailability is especially helpful when attempting to lose weight and to control hunger which can be stimulated in many ways.

In an illustrative embodiment of a fast undertaken utilizing hunger reduction according to the claimed invention, Table 2 is hereby provided to document bioidentical free D-BetaHydroxyButyric Acid consumed along with corresponding blood glucose and blood ketone levels in mmol/L as well as Glucose/Ketone Index (Glucose mmol/L/Ketone mmol/L) data during a twelve day water fast. In this illustrative embodiment, bioidentical free D-BetaHydroxyButyric Acid is orally consumed in 5 gram to 15 gram formats as needed to reduce hunger to make fasting possible. The fast included only water and D-BetaHydroxyButyric Acid with a palatability agent orally consumed. Traditionally a water only fast is extremely difficult to achieve for more than two or three days. The participant as detailed confirmed that such long term (twelve day) fasting would not be possible without hunger reduction from D-BetaHydroxyButyric Acid.

TABLE 2

| Date | Time | Ketone | Glucose | GKI | Free D-BHB | |
|---|---|---|---|---|---|---|
| 24-Jul | 7:14am | 1.6 | 5.2 | 3.3 | | |
| 24-Jul | 10:02am | 1.7 | 4.6 | 2.7 | 10 | g |
| 24-Jul | 10:45am | 2.5 | 4.8 | 1.9 | | |
| 25-Jul | 4:45am | 1.5 | 4.3 | 2.9 | 15 | g |
| 25-Jul | 8:00pm | 1.9 | 4.3 | 2.3 | | |
| 26-Jul | 7:20am | 1.6 | 5.2 | 3.3 | 10 | g |
| 26-Jul | 7:44am | 3.7 | 4.8 | 1.3 | | |
| 26-Jul | 7:54am | 3.6 | 4.4 | 1.2 | | |
| 26-Jul | 8:10am | 3.7 | 4.8 | 1.3 | | |
| 26-Jul | 2:24pm | 2.6 | 4.4 | 1.7 | 5 | g |
| 26-Jul | 2:50pm | 4.8 | 4.1 | 0.9 | | |
| 27-Jul | 5:25am | 3.8 | 3.6 | 0.9 | | |
| 27-Jul | 7:18am | 3.7 | 3.8 | 1.0 | 5 | g |
| 27-Jul | 7:35am | 4.7 | 4.5 | 1.0 | | |
| 27-Jul | 7:50am | 5.3 | 3.7 | 0.7 | | |
| 27-Jul | 8:49am | 4.2 | 3.6 | 0.9 | | |
| 27-Jul | 3:54pm | 6.6 | 2.9 | 0.4 | | |
| 27-Jul | 4:58pm | 4.9 | 3.3 | 0.7 | | |
| 28-Jul | 4:15am | 3.1 | 3.8 | 1.2 | | |
| 28-Jul | 7:12am | 3.8 | 4.3 | 1.1 | 5 | g |
| 28-Jul | 7:31am | 5 | 5 | 1.0 | | |
| 28-Jul | 7:45am | 5.1 | 4.4 | 0.9 | | |
| 29-Jul | 5:06am | 6.2 | 3.9 | 0.6 | | |
| 29-Jul | 6:55am | 5.6 | 3.5 | 0.6 | 5 | g |
| 29-Jul | 7:15am | 6.2 | 3.6 | 0.6 | | |
| 29-Jul | 7:32am | 7.2 | 3.2 | 0.4 | | |
| 29-Jul | 7:51am | 7.2 | 3.8 | 0.5 | | |
| 29-Jul | 8:07am | 6.6 | 3.5 | 0.5 | | |
| 29-Jul | 1:38pm | 5.9 | 2.9 | 0.5 | 5 | g |
| 29-Jul | 3:40pm | 6.5 | 2.7 | 0.4 | | |
| 29-Jul | 4:00pm | 5.6 | 3.4 | 0.6 | | |
| 29-Jul | 7:11pm | 3.8 | 3.9 | 1.0 | 5 | g |
| 29-Jul | 7:32pm | 4.7 | 3.7 | 0.8 | | |
| 30-Jul | 6:10am | 4.3 | 4.4 | 1.0 | 5 | g |
| 30-Jul | 6:39am | 6.9 | 3.8 | 0.6 | | |
| 30-Jul | 6:56am | 5.8 | 3.4 | 0.6 | | |
| 30-Jul | 10:50am | 3.6 | 4.2 | 1.2 | 5 | g |
| 30-Jul | 11:18am | 5.3 | 4 | 0.8 | | |
| 30-Jul | 1:31pm | 5.1 | 3.6 | 0.7 | | |
| 30-Jul | 4:54pm | 5.7 | 2.8 | 0.5 | | |
| 30-Jul | 5:10pm | 5.7 | 3.1 | 0.5 | | |
| 31-Jul | 4:36am | 3.3 | 3.9 | 1.2 | | |
| 31-Jul | 7:29am | 5.8 | 4.4 | 0.8 | 5 | g |
| 31-Jul | 7:44am | 5.9 | 3.9 | 0.7 | | |
| 31-Jul | 4:25pm | 6.4 | 3.6 | 0.6 | | |
| 1-Aug | 7:43am | 5.2 | 3.3 | 0.6 | | |
| 1-Aug | 8:05am | 5.1 | 4.3 | 0.8 | 5 | g |
| 1-Aug | 8:35am | 6.3 | 3.7 | 0.6 | | |
| 1-Aug | 12:29pm | 5.7 | 3.6 | 0.6 | | |
| 2-Aug | 10:26am | 5.7 | 4 | 0.7 | | |
| 2-Aug | 10:45am | 6.2 | 3.8 | 0.6 | 5 | g |
| 2-Aug | 11:05am | 7.2 | 3.4 | 0.5 | | |
| 2-Aug | 1:49pm | 5.8 | 4.7 | 0.8 | 5 | g |
| 2-Aug | 2:08pm | 6.7 | 4.3 | 0.6 | | |
| 2-Aug | 2:10pm | 6.4 | 4.1 | 0.6 | | |
| 3-Aug | 7:45am | 7.2 | 3.4 | 0.5 | 5 | g |
| 3-Aug | 8:02am | 5.3 | 4 | 0.8 | | |
| 3-Aug | 8:15am | 5.5 | 3.9 | 0.7 | | |
| 3-Aug | 4:16pm | 4.8 | 3.7 | 0.8 | 10 | g |
| 3-Aug | 4:51pm | 7.4 | 3.8 | 0.5 | | |
| 3-Aug | 5:18pm | 7.1 | 3.8 | 0.5 | | |
| 4-Aug | 6:00am | 4.6 | 3.9 | 0.8 | 10 | g |
| 4-Aug | 7:19am | 5.6 | 4.1 | 0.7 | | |
| 4-Aug | 4:13pm | 5.4 | 4 | 0.7 | 10 | g |
| 5-Aug | 7:26am | 4.3 | 4.4 | 1.0 | | |
| 5-Aug | 10:04am | 5.9 | 3.7 | 0.6 | | |

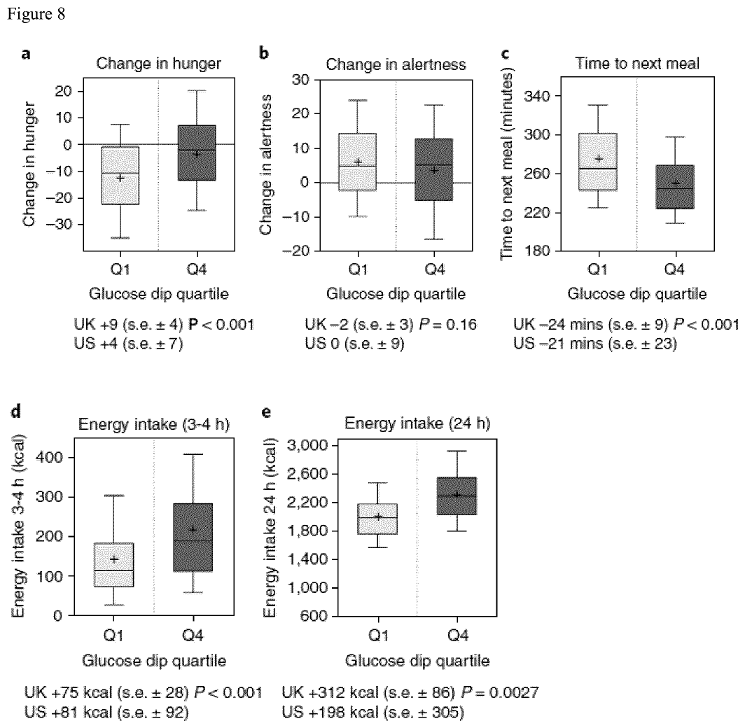

FIG. 8 is a graphical illustration of big dipper vs little dipper theory of glucose regulation and hunger. Food cues which are the sight or smell of food causing hunger. Social cues a friend's invitation to dinner for celebration or commiseration or pressure to eat certain foods can cause hunger. Stress cues having high levels of stress and exaggerated glucose spikes and troughs can lead to hunger. Essentially food cues, social cues and stress all elevate glucose leading to bigger changes in glucose and exaggerated hunger signals. (Yau 2013, Schuz 2015). Post prandial drops in glucose over 20 mg/dL will stimulate hunger and look like low glucose with high insulin and explains why cheese pizza tastes better two hours after you have eaten lunch than it does at lunch. This is also called the big dipper vs little dipper theory of glucose regulation and hunger. FIG. 8 is taken from Wyatt 2021 Natural Metabolism in which Big dippers are the dark blue on right and experience 10% more hunger, ate 24 minutes sooner and consumed 321 kcal more over 24 hours than their little dippers in light blue on left. Big dippers are the dark blue on right and experience 10% more hunger, ate 24 minutes sooner and consumed 321 kcal more over 24 hours than their little dippers in light blue on left. As the brain reward system prioritizes carbs+fat over carbs or fats. Consequently hunger signals drive individuals to eat bread with butter and not just a piece of bread or butter. (Cell Metabolism, 2018; DOI: 10.1016/j.cmet.2018.05.018). All of these triggers for hunger are tied to changes in energy regulation. Natural free D-BHB is an efficient fuel that provides quick energy and reduces the hunger signal and reduce caloric intake over the long term. It is an anticipated embodiment of the claimed invention that in addition to exigent D-BHB administration, follow-on D-BHB administration between 5 g and 30 g regularly administered over days, weeks, months and even years can improve resilience against hunger, achieve positive outcomes and enhance diet and metabolic recovery.

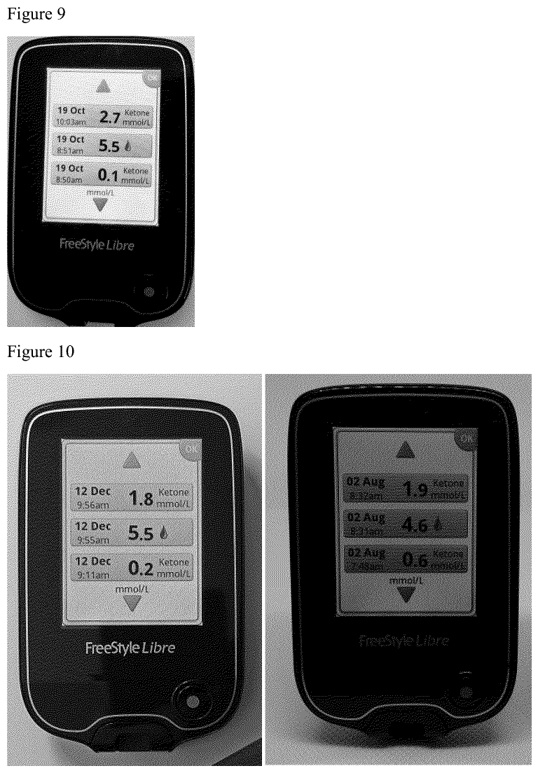

FIG. 9 is a graphical illustration of D-BHB exogenous ketone blood levels. The above Abbot blood ketone meter evidenced by Abbott blood test strip depicts the rapid rise in blood ketone levels from 0.1 mmol/L (absence of ketosis) to 2.7 mmol/L (ketosis) after oral bolus administration of 15 grams of bioidentical free D-BetaHydroxyButyric Acid. The ketone levels depicted are attained in the absence of a salt as well as without ester bonds. It is the free acid D-BHB exogenously administered which is bio-identical to the endogenous D-BHB free acid generated by the human body. The bioidentical ketone body D-BHB is first and foremost an energy source, which is directly and preferentially metabolized by the brain. D-BHB is also active as a signaling mechanism and hunger reduction agent, resulting in broad and beneficial anti-hunger physiological related benefits.

FIG. 10 is a graphical illustration of D-BHB exogenous ketone blood levels. FIG. 10 details rapid ketosis within 45 minutes confirmed by Abbott blood test strip after 10 g D-BHB with palatability agent administered by oral drink. Blood ketone levels document rise from 0.2 mmol/L to 1.8 mmol/L and 0.6 mmol/L to 1.9 mmol/L in a variety of test subjects. FIG. 10 reinforces the fact that supplementation by utilizing free D only BetaHydroxyButyric Acid without ketone salt or ketone ester is a reliable, reproducible method and system for rapid ketone level increase for hunger reduction and weight maintenance. While amounts of free D only BetaHydroxyButyric Acid taken for hunger effects may vary, it is an anticipated preferred embodiment that levels between 5 and 30 grams of D only BetaHydroxyButyric Acid are taken as needed for optimum hunger reduction. Alternate embodiments may include lower or higher amounts, however, experimental evidence has confirmed optimal administration in this range for a wide variety of other applications.

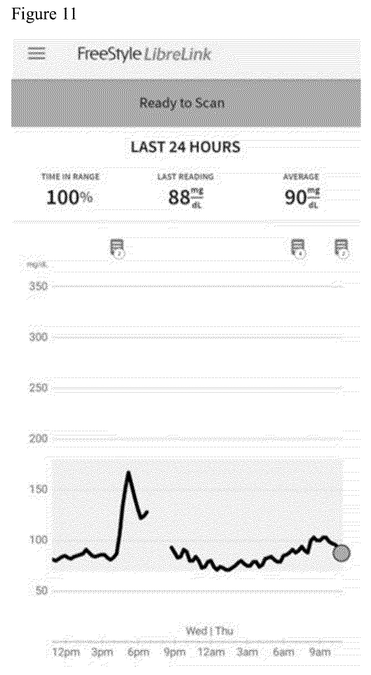

FIG. 11 is a graphical illustration of continuous monitoring of body glucose levels. FIG. 11 depicts continuous glucose monitoring levels with carbohydrate challenge documented by Abbot Libre Continuous Glucose Monitoring (CGM) device. In this illustrative embodiment, glucose levels from a 50 g carbohydrate ingestion are depicted unaided by free D-BetaHydroxyButyric Acid on the left and repeated the following day with two 50 g carbohydrate ingestions accompanied by 10 g free D-BetaHydroxyButyric Acid administered 45 minutes before the carbohydrate insult. The data is noteworthy in that while the equivalent amount of identical carbohydrates were consumed, the rapid glucose spike depicted in the unsupplemented ingestion is not found with 10 g D-BetaHyrdoxyButyric Acid accompaniment. As free D-BetaHydroxyButyric Acid can lower glucose while rapidly increasing ketone levels, it is believed that obtaining high ketones while lowering glucose will yield additional hunger reduction benefits.

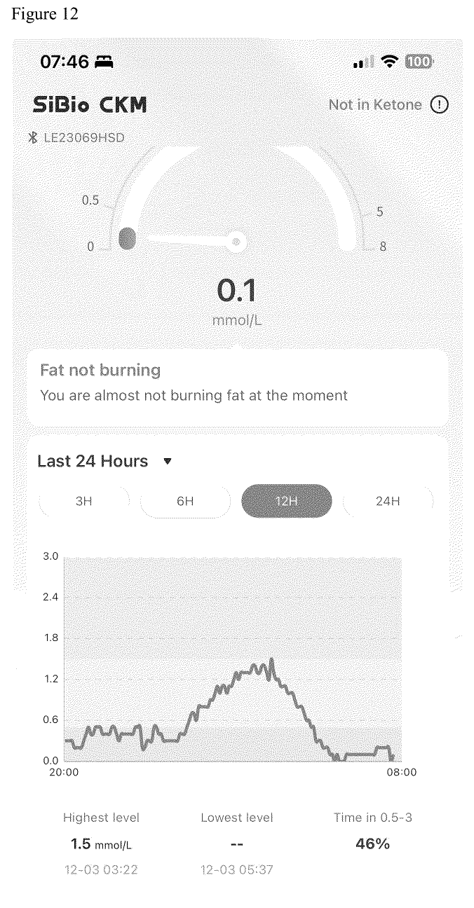

FIG. 12 is a graphical illustration of continuous monitoring of body ketone levels resulting in improved hunger reduction due to enhanced D-BHB levels according to the claimed invention. It is noteworthy that consumption of even small amounts of free D-BetaHydroxyButyric Acid can give rise to increased ketosis levels as measured by Continuous Ketone Meter (CKM). In this representative example, 5 grams of free D-BetaHydroxyButyric Acid consumed 45 minutes prior to bedtime resulted in a 1.5 mmol/L increase can also enhance the quantity of deep sleep obtained via hunger reduction. While free D-BetaHydroxyButyric Acid is not a sedative, even small amounts can greatly improve hunger reduction.

### II. D-Beta HydroxyButyric Acid Hunger Reduction Method & System Disclosure

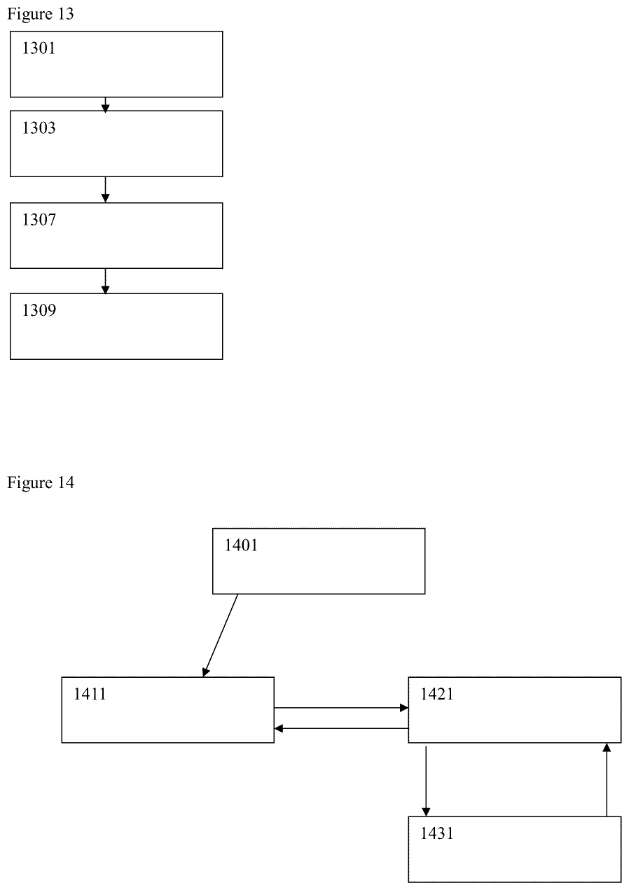

FIG. 13 is a schematic illustration of a preferred bioidentical D-BetaHydroxyButyric Acid hunger reduction protection. In a preferred embodiment, a method of hunger management is obtained through the steps of:
Assessing (1301) ketone levels and hunger symptoms,
Administering (1303) a beneficial amount of free D-BetaHydroxyButyric acid,
Evaluating (1307) the hunger symptoms of a subject in need, and
Readministering (1309) free D-BetaHydroxyButyric acid.

FIG. 14 is a schematic illustration of a preferred D-BHB hunger management system. In the illustrative embodiment, ketone level monitor (1421) determines the amount of free D-BetaHydroxyButyric Acid for a hunger subject in need thereof, optionally and additionally informed by glucose monitor (1433). After Free D-BetaHydroxyButyric Acid initial dose (1401) is administered, Free D-BetaHydroxyButyric Acid supplemental dose (1411) is administered.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments. In particular, free D-BetaHydroxyButyric Acid may be administered diluted or with a preferred palatability agent without detracting from the spirit or scope of the claimed invention.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

I claim:

1) An enhanced hunger reduction method comprising the steps of:
Determining metabolic wellness of intended patient,
Administering D-BetaHydroxyButyric Acid compound orally consumed in a physiologically suitable frequency to enhance hunger reduction,
Evaluating the hunger symptoms of a subject in need, and
Readministering free D-BetaHydroxyButyric acid until hunger symptoms are mitigated.

2. The method of claim 1 wherein said Administering an effective amount of free D-BetaHydroxyButyric Acid is provided in a 5 g-30 g net D-BetaHydroxyButyric Acid format.

3. The method of claim 2 wherein said free D-BetaHydroxyButyric Acid additionally comprises a ketogenic complementary nutritional supplement selected from the group consisting of akkermansia, taurine, collagen, creatine and thiamine.

4. A hunger management system comprising a ketone level monitor which determines the amount of free D-BetaHydroxyButyric Acid for a hunger subject in need thereof, a glucose monitor and Free D-BetaHydroxyButyric Acid effective dose.

5. The hunger management system of claim 4 wherein the amount of free D-BetaHydroxyButyric Acid administered is between 5 grams and 30 grams of free D-BetaHydroxyButyric acid.

6. The system of claim 5 wherein the ketone level monitor which determines the amount of free D-BetaHydroxyButyric Acid for a hunger subject in need thereof additionally comprises functionality for regular delivery of free D-BetaHydroxyButyric Acid until hunger symptoms are substantially mitigated.

7. A hunger mitigation compound comprising free D-BetaHydroxyButyric Acid administered in 5 g-30 g format in conjunction with a ketogenic complementary nutritional supplement selected from the group consisting of akkermansia, taurine, collagen, creatine and thiamine.

8. The compound of claim 7 wherein said ketogenic complementary nutritional supplement additionally comprises a palatability agent incorporating sugar free sweetener selected from the group consisting of stevia and allulose as well as natural fruit essence distillate as a palatability enhancer.
